(19) United States
(12) Patent Application Publication
(10) Pub. No.: US 2004/0265345 A1
(43) Pub. Date: Dec. 30, 2004

(54) Treatment of skin damage using acetyl carnitine and lipoic acid

(76) Inventor: Nicholas V. Perricone, Guilford, CT (US)

Correspondence Address: ST. ONGE STEWARD JOHNSTON & REENS, LLC, 986 BEDFORD STREET, STAMFORD, CT, 06905-5619, US

(21) Appl. No.: 10/609,777

(22) Filed: Jun. 30, 2003

(51) Int. Cl. A61K 31/385; A61K 31/205; A61K 31/375; A61K 31/198

(52) U.S. Cl. 424/401; 514/440; 514/554; 514/474; 514/557; 514/567

(57) ABSTRACT

A composition containing both acetyl carnitine and lipoic acid are topically applied to treat skin damage, such as contact dermatitis, atopic dermatitis, xerosis, eczema, rosacea, seborrhea, psoriasis, thermal and radiation burns, other types of skin inflammation, and aging. Typical compositions contain from about 0.025% to about 5%, more narrowly from about 0.5% to about 2% by weight acetyl carnitine, and from about 0.1% to about %, more narrowly from about 0.25% to about 5% lipoic acid or lipoic acid derivative in a dermatologically acceptable carrier that contains phosphatidylcholine. Many embodiments also contain at least one adjunct ingredient such as tyrosine, a fatty acid ester of ascorbic acid such as ascorbyl palmitate, a α-hydroxy acid such as glycolic acid, and/or folic acid. A preferred embodiment contains acetyl carnitine, lipoic acid, and tyrosine.

### STATEMENT REGARDING FEDERALLY SPONSORED RESEARCH

Not Applicable

### BACKGROUND OF THE INVENTION

1. Field of the Invention

This invention relates to the topical application of acetyl-carnitine and lipoic acid for the treatment of acute and chronic skin damage. Therapies according to the invention are particularly efficacious for treating a variety of skin conditions, including dermatitis, xerosis, eczema, rosacea, seborrhea, psoriasis, thermal and radiation burns, other types of skin inflammation, and the tissue degenerative effects of aging.

2. Description of Related Art

Skin inflammation and aging are closely related phenomena. So similar are the processes involved with both, that aging is sometimes described dermatologically as a chronic low grade inflammatory condition. In acute inflammation, there is typically a respiratory burst of neutrophil activity that initiates cascades that typically involve a change in the oxidation state of the cell. Acute inflammation is also characterized by mast cell degranulation wherein serotonin is produced, which acts as a signal transduction factor. Following that, excited oxygen species are generated, e.g., superoxide anion, and these damage the lipid-rich membranes and activate the chemical mediators of proinflammation and inflammation.

Alteration in the redox state of the cell activates transcription factors such as NFκB as well as AP1, which then causes production of proinflammation mediators. These mediators, such as TFα and various interleukins, cause a burst of cytokines. Arachadonic acid is released, which is oxidized to biologically active mediators. When arachadonic acid is oxidized via the cyclooxygenase or lipoxygenase pathways, for example, prostaglandins, leukotrines, and hyroxyeicosatetraenoic acid (HETE) are produced, which cause erythma, edema, and free radical production. Transcription factors such as NFκB and AP1 alter DNA expression in the cell and produce cytokines and proteinases such as collagenase. Thus, in the long run, stimulation of the inflammation cascade causes more damage than that caused by the initial free radicals.

Similar metabolic events are observed in skin aging. Cell age is due in part to free radical damage, which takes place mostly within the cell membrane. The cell membrane is most susceptible to attack by free radicals because of its dense molecular structure largely comprising lipids and lipoproteins, which are easily oxidized by reactive oxygen species. In skin, reactive oxygen species such as singlet oxygen, the superoxide anion, and hydroxyl radicals, as well as other free radicals, are generated in normal metabolism, as well as through ultraviolet sun exposure, other forms of radiation, other environmental factors such as pollution or exposure to chemicals in the home or workplace, and the like, active in the arachidonic acid cascade. As in inflammation, free radicals activate chemical mediators that produce prostaglandins and/or leukotrines, stimulating the inflammation cascade.

The body contains an endogenous antioxidant defense system made up of antioxidants such as vitamins C and E, glutathione, and enzymes, e.g., superoxide dismutase. When metabolism increases or the body is subjected to other stress such as infection, extreme exercise, radiation (ionizing and non-ionizing), or chemicals, the endogenous antioxidant systems are overwhelmed, and free radical damage takes place. Over the years, the cell membrane continually receives damage from reactive oxygen species and other free radicals, resulting in cross-linkage or cleavage or proteins and lipoproteins, and oxidation of membrane lipids and lipoproteins. Damage to the cell membrane can result in myriad changes including loss of cell permeability, increased intercellular ionic concentration, and decreased cellular capacity to excrete or detoxify waste products. As the intercellular ionic concentration of potassium increases, colloid density increases and m-RNA and protein synthesis are hampered, resulting in decreased cellular repair. Some cells become so dehydrated they cannot function at all.

In skin aging, the regularity of tissue structure is lost. Individual cells enlarge, but the total number of cells decreases approximately 30%. Intercellular collagen increases, and the proportion of soluble collagen decreases. Cross-linking between long-chain collagen macromolecules occurs. Elastin loses its discrete structure and elasticity, and has an increased calcium content. The dermis microscars and diminishes.

Sunlight and chemical exposure wreaks far greater destruction on the skin than time itself, and intensifies and augments the aging process. There is substantial evidence that ultraviolet radiation induces the formation of reactive oxygen species which are implicated as toxic intermediates in the pathogenesis of photoaging (Ibbotson, S. H., et al., J. Investig. Derm. 112: 933-938 (1999)). Activation of transcription factors such as AP1 causes gene expression of collagenases which cause further damage. Free radical damage to the surface of the skin from sun and chemical exposure is manifested as lines, mottling, discoloration, precancers and cancers. Aging of both skin and other tissues is, in part, the result of constant free radical damage to cell membranes, leading to decreased cell function. This results in accumulation of waste products in the cells, such as lipofuscin; increase in the potassium content of the cells, which results in dehydration of the cells; and decreased production of messenger RNA and proteins.

Many formulations for treating inflammation and aging effects in skin are steroids or lubrications and emollients that are simply topical compositions containing soothing agents, e.g., as exemplified by commercial hand lotion products and the like. More recently, attention has been directed to agents which address the underlying processes involved in skin damage, such as the free radical generation processes (N. V. Perricone, The Wrinkle Cure, Rodale Press, Emmaus, Pa., 2000). In this regard, investigations have been made with respect to the antioxidants vitamin E and vitamin C to quench free radicals on the surface of the skin and to protect lipid membranes intracellularly (Wilson, R., Drug and Cosmetic Industry, 32-34, 38, and 68, August 1992).

It would be desirable to have alternative topical compositions for skin damage and aging, particularly compositions that are efficient in free radical scavenging intracellularly and in membranes, and in inhibiting the inflammation cascade.

### BRIEF SUMMARY OF THE INVENTION

It is an objective of the invention to provide new methods for the treatment of skin damage, such as atopic dermatitis, contact dermatitis, xerosis, eczema, rosacea, seborrhea, psoriasis, thermal and radiation burns, other types of skin inflammation, as well as aging.

These and other objectives of the invention are accomplished by the present invention, which provides methods for topically applying compositions containing a synergistic combination of acetyl carnitine and lipoic acid to exposed or affected skin areas, primarily for the treatment but also for the prevention of skin damage, often in association with a dermatologically acceptable carrier such as one containing phosphatidylcholine. The amount of acetyl carnitine and lipoic acid necessary to treat damaged skin is not fixed per se, and necessarily is dependent upon the amounts and proportions of the two active ingredients in the preparation, the amount and identity of any adjuct ingredients such as tyrosine, folic acid, fatty acid esters of ascorbic acid, e.g., ascorbyl palmitate, and/or α-hydroxy acids, e.g., glycolic acid, the dermatological carrier such as one containing phosphatidylcholine selected, the user's skin type, and the severity, extent, and nature of the dermatological problem treated. In some typical embodiments, the composition contains from about 0.025% to about 5 weight %, more narrowly from about 0.5% to about 2%, by weight acetyl carnitine, and, and from about 0.5% to about 7%, more narrowly from about 1% to about 5% by weight lipoic acid.

### DETAILED DESCRIPTION OF THE INVENTION

This invention is based upon the surprising finding that acetyl carnitine and lipoic acid act synergistically when used together in topical compositions for the treatment of skin damage and aging.

Acetyl carnitine has in the past been suggested for the treatment of viral infections, including skin infections such as those caused by Herpes, including lesions that occur after sunlight or UV exposure (U.S. Pat. No. 5,314,689 to Scandurra and Aurelian). Acetyl carnitine was suggested as an optional ingredient in a composition containing glutathione and selenoamino acid for reducing and repairing x-ray radiation-induced skin damage (U.S. Pat. No. 5,667,791 to Hersh and Warshaw). Oral administration of acetyl carnitine has also been suggested for the treatment of AIDS symptoms, including skin inflammation (U.S. Pat. No. 5,667,791 to Weil and Scandurra). Oral or parenteral acetyl carnitine has also been disclosed as useful in stimulating the immune system of patients with an impaired immune system (U.S. Pat. No. 4,415,588 to Cavazza), and in the therapeutical treatment of impaired cardiac function, myocardial anoxia and cardiac arrhythmias (ibid.).

Lipoic acid was originally identified as a bacterial growth factor present in the water-soluble fraction of liver and yeast. It was found to be necessary for the oxidative decarboxylation of pyruvic acid by Streptococcus fecalis and for the growth of Tetrahymena gelii, and replaced acetate for the growth of Lactobacillus casei. Subsequent research showed that lipoic acid was a growth factor for many bacteria and protozoa, and it served as a prosthetic group, coenzyme, or substrate in plants, microorganisms, and animal tissues, participating in a variety of metabolic processes including acyl transfer reactions unction determined that it is a co-factor for α-keto-dehydrogenase complexes. Its reduced form, dihydro-lipoic acid (herein sometimes referred to as DHLA), is a potent sulfhydryl reductant. In aqueous systems, both lipoic acid and DHLA exhibit antioxidant actions (reviewed by Packer, L., et al., Free Rad. Biol. Med., 1995, 19: 227-250 (1995)). Lipoic acid has been shown to maintain microsomal protein thiols, protect against hemolysis, and protect against neurological disorders. The protective effect of dietary supplementation of lipoic acid against ischemia/reperfusion injury in the Langendorff isolated heart model has also been demonstrated. Lipoic acid has been suggested for treating systemically, or as adjuvant systemic medication for, liver cirrhosis, atheroschlerosis, diabetes, neurodegenerative diseases, heavy metal poisoning, mushroom poisoning and Chagas disease.

A couple of recent references have suggested that lipoic acid might be useful in dermatological compositions. In a 1988 Japanese patent publication (JP 63008315), lipoic acid in cosmetics at concentrations of 0.01% to 1%, preferably 0.05% to 0.5%, or in topical “quasi-drugs” at concentrations of 0.1% to 1.5%, preferably 0.5% to 1.0%, were suggested for inhibiting tyrosinase, and thus melanin formation, to whiten skin.

In 1995, Rawlings, et al., disclosed a composition and method for “improving or preventing the appearance of dry, flaky wrinkled, aged, photo-damaged skin and treating skin disorders” (U.S. Pat. No. 5,472,698, column 2, lines 51 to 54) using a synergistic combination of serine and/or N-acetyl serine and a thiol, an “S-ester”, and/or a disulfide (id., lines 28 to 33). Lipoic acid was mentioned as encompassed by the latter ingredient (column 3, lines 29 to 30), but the terminology in the patent was confusing because thiols and S-esters were disclosed as preferable over disulfides such as lipoic acid. And the focus of the patent was stimulation of sphingolipid synthesis in skin, with lipoic acid shown to be the same as control compositions, and have no effect in combinations without serine.

A year later, Weischer, et al., (in U.S. Pat. No. 5,569,670), pharmaceutical compositions containing a synergistic combination of α-lipoic acid and/or dihydrolipoic acid with specific enantiomers of these, together with some vitamins, including C and E (column 1, lines 3 to 15), were disclosed as useful, primarily for treating diabetes (see the claims). However, anti-inflammatories (abstract, line 8 and column 2 at line 16) as well as treatments for retroviruses and other pathological conditions were included, with an emphasis on veterinary applications (column 13, lines 42 to 62). In a test model for inflammation (observing rat edema), the R-enantiomer of lipoic acid was superior to lipoic alone or to vitamin E alone (column 3, lines 37 to 40). Suggested administration was oral, parenteral or intravenous (column 7, line 31 to end, et seq.), preferably oral (column 11, line 42), but application to skin and mucous membranes was mentioned (column 12, lines 58 to 60). An ointment was disclosed in Example 6; the others described suppositories, capsules, ampules, and tablets.

Similarly, U.S. Pat. No. 5,693,664 to Wessel, et al., from the same research group, was directed to diabetes treatments, particularly where insulin resistance is observed (column 1, lines 10 to 14 and the claims) by use of the R-enantiomer of α-lipoic acid. Again, one enantiomer, not a racemate, was employed (column 6, lines 18 to 19). Indeed, the S-enantiomer decreased the effect of insulin in an experimental study reported (column 3, lines 61 to 65). Suggested adminstration was primarily oral (column 6, lines 61 to 66), though parenteral and intravenous are mentioned (ibid., and column 3, lines 7 to 8).

U.S. Pat. No. 5,728,735 to Ulrich, et al., again from the same group, stressed use of an enantiomer (column 1, lines 28 to 54), particularly the R-enantiomer (see the claims), and not a racemate, for combatting pain and inflammation in a variety of conditions (id., lines 58 to 59; inflammations are listed in column 5, line 64 to column 6, lines 9 and include neurodermatitis and psoriasis). Suggested administrations were oral, intravenous, or infusions (column 3, lines 28 to 30, 51, 62 to 63 and 65), but solutions and emulsions for topical application were mentioned (column 6, lines 29 to 34 and 65 to 68, and column 8, lines 16 to 18). Only tablets and ampules were illustrated. All the 2 5 reported findings of the group are complicated by the fact that the metabolic effects of the R- and S-enantiomers are now known to be different, as are the enzymes that process the enantiomers in cytosolic and mitrochondrial systems (Haramaki, N., et al., Free Rad. Biol. Med. 22: 535-542 (1997)). Moreover, different stereospecific reduction by intact cells and tissues has also been 3 0 observed (ibid.). More recently, Perricone suggested the use of lipoic acid in dermatological compositions for the treatment of skin damage, particularly inflammation and aging (U.S. Pat. No. 5,709,868) and acne (U.S. Pat. No. 6,365,623), and also for the treatment of scars, particularly for hypertrophic and keloid scars (U.S. Pat. No. 5,965,618).

In the practice of the invention, acetyl carnitine and lipoic acid are used together to formulate especially efficacious topical compositions for treating a variety of pathological skin conditions and aging. L-acetyl carnitine and/or biologically equivalent derivatives of acetyl carnitine such as D-acetyl cartinine, DL-acetyl carnitine, DL-acetyl cartinine hydrochloride, DL-acetyl carnitine chloride, acetyl D,L carnitine hydrochloride, acetyl L-carnitine hydrochloride can, when administered with together with lipoic acid, often in association with a dermatologically acceptable carrier and adjunct ingredients more fully described below, penetrate skin and cause enhanced function of skin cells and skin structures such as keratinocytes and fibroblasts. Advantageous embodiments deliver carnitine directly into skin cells through the use of acetyl carnitine and/or a biologically active derivative, to enhance mitochondrial function. This, in turn, leads to high energy production in the cell, which then can maintain the integrity of cellular membranes and increase exchange of nutrients and wastes across these membranes. Use of effective amounts of acetyl carnitine together with lipoic acid gives skin a softer, smoother appearance, and decreases epidermal age pigments.

Any synthetic or natural acetyl carnitine preparation may be employed in compositions of the invention. Acetyl carnitine,

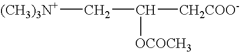

is a known compound that can be purchased, prepared, or isolated from natural sources. As illustrated above, it contains an asymmetric carbon atom, and thus exists in the racemic form and in the optically active dextrorotary and levorotary forms. As used herein, the term “acetyl carnitine” encompasses D-, L-, or DL-acetyl carnitine, and biologically active derivatives such as those listed above. L-acetyl cartine is preferred because it is the most active form. Preferred concentrations of acetyl carnitine in compositions of the invention vary from about 0.025% to about 5%, more narrowly from about 0.5% to about 2%, by weight.

The term “lipoic acid” encompasses thioctic acid (1,2-dithiolane-3-pentanoic acid; 1,2-dithiolane-3-valeric acid), C₈H₁₄O₂S₂, formula weight 206.32, and its reduced form, dihydrolipoic acid. It has been variously known as acetate replacing factor, protogen A, and pyruvate oxidation factor. As mentioned above, for convenience, as used herein, where the properties and advantages of “lipoic acid” (or LA) are discussed as an active ingredient in the practice of the invention, both lipoic acid and its derivatives are encompassed. “Lipoic acid derivatives” include thioctic acid esters, particularly alkyl esters such as fatty acid esters, amides, particularly those isolated from or mimicking naturally occurring lipoamides, salts, particularly alkali metal salts, anhydrides and specifically includes the reduced form, dihydrolipoic acid and its esters, amides and salts. One particularly efficacious derivative that exhibits increased cellular uptake and biological activity useful in the practice of the invention is N,N-dimethyl,N-2-amidoethyl lipoate recently described by Sen, C. K., et al. (Free Radical Biol. Med., 1998, 25: 89) and called lipoic acid plus (LA-Plus). Since lipoic acid is both fat- and water-soluble, it is an advantage of the invention that it can be used in either lipid or aqueous-based compositions, and it readily crosses cellular membranes and disperses in extracellular and intracellular tissue components. Derivatives may also include those involving other reactive groups known to those skilled in the art. As used herein, the term “derivatives” includes metabolic precursors of lipoic acid. Where lipoic acid derivatives are employed, they must be functionally equivalent to lipoic acid.

As described more fully below, the active ingredient combination is topically applied to exposed or affected skin areas in amounts effective to treat skin damage. By “effective amount” is meant an amount of both active ingredients sufficient to stabilize the cell plasma membrane by scavenging and neutralizing free radicals and exhibiting antioxidant activity, thereby inhibiting the arachidonic acid cascade which leads to the activation of transcription factors that direct the cell nucleus into producing pro-inflammatory chemicals such as arachidonic acid. In the practice of the invention, active ingredients are typically delivered to lipid-rich layers of the skin in amounts effective to prevent inflammation and accelerate collagen synthesis.

Some embodiments of this invention contain at least one other adjunct ingredient such as tyrosine in addition to acetyl carnitine and lipoic acid. Compositions of the invention that comprise tyrosine typically are formulated to contain from about 0.01% to about 6%, more narrowly from about 0.03% to about 5% by weight, and, in many embodiments, from about 0.2% to about 3% by weight tyrosine, based on the total composition. Preferred formulations contain from 0.1 to 5% by weight tyrosine.

Alternate or additional adjunct ingredients include α-hydroxy acids. As used herein, the term “α-hydroxy acid” has reference to and encompasses the general class of organic compounds containing at least one hydroxy group and at least one carboxyl group, and wherein at least one hydroxyl group is located on the α-carbon atom. Typically, the compounds are organic acids having at least one carboxylic acid group and at least one hydroxyl group on the α-carbon atom, and. may contain other functional groups including additional hydroxyl and carboxylic acid moieties. Preferred α-hydroxy acids and/or α-hydroxy acid derivatives are less bulky structurally so that they penetrate the skin well, and thus have a backbone of from one to three carbon atoms such as those set out in U.S. Pat. No. 5,965,618 at column 6 lines 4 to 29. Where employed, glycolic and/or lactic acid or their derivatives are preferred; glycolic acid is especially efficacious. Glycolic acid or other α-hydroxy acids are typically present in amounts ranging from about 1% to about 10%, more narrowly from about 3% to about 7% of the total composition.

Fat-soluble fatty acid esters of ascorbic acid (vitamin C) are employed as an adjunct ingredient in other embodiments, alone or in combination with tyrosine, and/or α-hydroxy acids and/or folic acid described below. The more oxidation-resistant saturated fatty acid esters of ascorbic acid are preferred, including, but not limited to, ascorbyl laurate, ascorbyl myristate, ascorbyl palmitate, ascorbyl stearate, and ascorbyl behenate. As is known by skilled workers, ascorbic acid esters include mono-, di-, tri- and tetraesters, and mixtures thereof. Ascorbyl palmitate is used in one embodiment. As denoted herein, where fatty acid esters are described, e.g., ascorbyl stearate, compositions having predominantly that ester, e.g., predominantly stearate, are included. The esters may be prepared using hydrogenated oils or fats, or fractions thereof, and contain small amounts of another ester. Ascorbyl stearate prepared using canola, for example, commonly contain about 4% ascorbyl palmitate. It is an advantage of the invention that where fatty acid esters of ascorbic acid are employed as an adjunct ingredient, they help stabilize the alkanolamine in the composition. Ascorbyl palmitate and the like ascorbyl esters are typically present in amounts ranging from about 0.5% to about 15%, preferably from about 1 % to about 7% to 10%, of the total composition. Vitamin A or vitamin A derivatives may be alternative or additional adjunct ingredients in like concentrations. Vitamin A and vitamin A derivates include, but are not limited to, retinol, retinyl palmitate, retinoic acid, retinal, and retinyl propionate.

As mentioned above, another adjunct ingredient useful in some compositions is folic acid. By “folic acid” is meant N-[4-[[(2-amino-1,4-dihydro-4-oxo-6-pteridinyl)methyl]amino benzoyl]-L-glutamic acid, also sometimes called pteroylglutamic acid, N-p-[[(2-amino-4-hydroxy-6-pteridinyl)methyl]amino]benzoyl]-glutamic acid, or N-(p-[(2-amino-4-hydroxypyrimido[4,5-b]pyrazin-6-yl)methylamino]benzoyl-glutamic acid. Physiological salts of folic acid such as potassium or sodium salts and simple C₁ to C₄ esters may also be employed, provided that they exhibit the biological properties of folic acid. As used herein, when the term “folic acid” is used, it encompasses biologically equivalent derivatives. Typical folic or folate concentrations range between about 0.025% to about 1% by weight, more narrowly from about 0.05% to about 0.5% by weight.

Only effective amounts of acetyl carnitine and lipoic acid are needed to provide observable improvement in skin, alone, or in combination with adjunct ingredients, so generally topical application is accomplished in association with a carrier, particularly where lower amounts are needed to treat skin damage (including either inflammation or aging or both), or where the carnitine, lipoic, and adjunct ingredients not effectively solubilized. Where employed, the carrier is inert in the sense of not bringing about a deactivation or oxidation of the acetyl carnitine or lipoic acid and in the sense of not bringing about any adverse effect on the skin areas to which it is applied. Where carriers or vehicles (e.g., lotions, creams, ointments, soaps, sticks, or the like) are employed, they are formulated as to facilitate topical application and, in some cases, provide additional therapeutic effects as might be brought about, e.g., by moisturizing of the affected skin areas. While the carrier for dermatological compositions can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration and/or aid in the percutaneous delivery of the active agent. Many preparations are known in the art, and include lotions containing oils and/or alcohols and emollients such as hydrocarbon oils and waxes, silicone oils, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, as well as lecithin, and generally also emulsifiers (nonionic, cationic or anionic), although some such as lecithini inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. Such compositions are referred to herein as dermally or dermatologically acceptable carriers.

Suitable carriers include water, alcohols, oils and the like, chosen for their ability to dissolve or disperse acetyl carnitine and lipoic acid, and any other ingredients used in the treatment. Generally, even low concentrations of active ingredients in a carrier are suitable, depending upon the application regimen and adjunct ingredients employed. Chronic conditions typically require a lower concentration of active acetyl carnitine ingredient than to acute conditions. As a practical matter, however, to avoid the need for repeated application, it is desirable that the topically applied compositions be formulated to contain at least about 0.01% by weight, preferably at least about 0.025% acetyl carnitine and at least about 0.01%, preferably at least about 0.025% lipoic acid. As summarized above, typical compositions of the invention contain from about 0.5% to about 2% acetyl carnitine and from about 1% to about 5% lipoic acid. Generally in the practice of methods of the invention, the composition is topically applied to the affected skin areas in a predetermined or as-needed regimen either at intervals by application of a lotion or the like, it generally being the case that gradual improvement is noted with each successive application. Insofar as has been determined based upon clinical studies to date, no adverse side effects are encountered.

Many preferred embodiments employ a phosphatidylcholine carrier. Phosphatidylcholine (sometimes hereafter abbreviated “PC”), commonly called lecithin, is a mixture of diglycerides of stearic, palmitic, and oleic acids, linked to the choline ester of phosphoric acid. It can be isolated from eggs, soybeans, and other biological materials rich in PC, chemically synthesized, or obtained commercially from many sources. Food grade lecithin is preferred. Some commercial grades, for example, contain about 2.2% PC. It is an advantage of the invention that both acetyl carnitine, lipoic acid and phosphatidylcholine are natural products that have been shown to have no toxicity to mammals. Indeed, phosphatidylcholine is edible and digestible, and used in margarine, chocolate, and the food industry in general. Moreover, since acetyl carnitine penetrates poorly when topically administered, it is a particular advantage of the invention that combining it with lipoic acid and phosphatidyl choline considerably enhances penetration of this active ingredient.

Topical compositions of the invention can comprise additional ingredients commonly found in skin care compositions and cosmetics, such as, for example, tinting agents, tinting agents, emollients, skin conditioning agents, emulsifying agents, humectants, preservatives, antioxidants, perfumes, chelating agents, etc., provided that they are physically and chemically compatible with other components of the composition. Preservatives include, but are not limited to, C₁-C₃ alkyl parabens and phenoxyenthanol, typically present in an amount ranging from about 0.5% to about 2.0% by weight percent, based on the total composition. Emollients, typically present in amounts ranging from about 0.01 % to 5% of the total composition include, but are not limited to, fatty esters, fatty alcohols, mineral oils, polyether siloxane copolymers, and mixtures thereof. Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Emulsifiers, typically present in amounts from about 1% to about 10% by weight of the composition, include, but are not limited to, stearic acid, cetyl alcohol, stearyl alcohol, steareth 2, steareth 20, acrylates/C₁₀₋₃₀ alkyl acrylate crosspolymers, and mixtures thereof. Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof. Antioxidants, typically present in an amount ranging from about 0.02% to about 0.5% by weight of the composition, include, but are not limited to, butylated hydroxy toluene (BHT); vitamin C and/or vitamin C derivatives, such as fatty acid esters of ascorbic acid, particularly asocorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these. As mentioned above, particularly preferred antioxidants are those that provide additional benefits to the skin such as ascorbyl palmitate.

Buffering agents are employed in many compositions. Preferably, the amount of buffering agent is one that results in compositions having a pH ranging from about 4.5 to about 8.5, more preferably from about 5.5 to about 8.5, most preferably from about 6.5 to about 8.0. Typical buffering agents are chemically and physically stable agents commonly found in cosmetics, and can include compounds that are also adjunct ingredients such as citric acid, malic acid, and glycolic acid buffers.

While not wishing to be bound to any theory, it is possible that acetyl carnitine and lipoic acid are efficacious in the treatment of skin damage because they readily disperse in cell membranes and other cellular components. Compositions of the invention readily penetrate skin. Indeed, since lipoic acid is both water-and fat-soluble, it is sometimes referred to as a universal antioxidant. Both active ingredients are antioxidants that have been shown to protect against lipid peroxidation. They also act as free radical scavengers and neutralizers, particularly against superoxide anions, and prevent the cross-linking of cell membranes that is often seen in its post-inflammatory phases. By the same token, modulation of free radicals and other oxidative species by acetyl carnitine and lipoic acid appears to affect gene expression, including expression of nuclear factor κ-B (NF-κB), nitric oxide synthetase and other mediators at all stages of proinflammation and inflammation. The alteration of lipid peroxidation, protein cross-linking, growth factor stimulation, and membrane permeability may explain their negative effect on the symptoms of damaged skin. And the effects of the active ingredients are enhanced by using them in combination with tyrosine, at least one α-hydroxy acid such as glycolic acid, ascorbyl fatty acid esters such as ascorbyl palmitate, and folic acid because these compounds also act as antioxidants and scavenge free radicals.

When skin is inflamed from ultraviolet radiation, irritants, trauma, and other reasons, phospholipase-A-2 produces arachidonic acid from the phospholipid-rich membranes of the cell, resulting in the production of metabolites. We now know that stabilization of the cell membrane can inhibit the inflammatory cascade, therefore preventing the inflammatory response. It is also now known that arachidonic acid has a direct toxic effect on the mitochondria, resulting in the uncoupling of oxidative phosphorylation, resulting in free radical damage to the mitochondrial membrane. Acetyl carnitine and lipoic acid appear to intersperse in the cell membrane, stabilizing the membrane, and, at the same time, providing antioxidant capability and reenforcing one another.

Acetyl carnitine acts to suppress an increase in reduced glutathione and reduced ubiquinone levels, providing a stabilizing effect on membranes by decreasing membrane lipid peroxidation. Carnitine is essential for the transportation of fatty acids across the mitochondrial membrane to be oxidized. It is known that carnitine enhances mitochondrial energy production, probably by increasing cytochrome oxidase activity, the final enzyme in the cellular respiratory chain. This results in a more efficient cellular maintenance and repair. In addition, the delivery of acetyl carnitine into the cell membrane appears to enhance membrane activity, such as exchange of nutrients and wastes of the cellular environment. This also enhances cellular function and repair. Lipoic acid likewise exhibits activity within the pyruvate dehydrogenase complex to such an extent that it has been termed the metabolic antioxidant. Again, while not wishing to be bound to any theory, together these ingredients appear to boost energy production, contributing significantly to the overall therapeutic effect of using compositions of the invention.

Methods and compositions of the present invention are particularly useful for treating damaged skin tissue, particularly various types of dermatitis, skin conditions such as rosacea, seborrhea, eczema, xerosis (dry skin), psoriasis, thermal and radiation burns, and other types of inflammation. Compositions of the invention are useful in treating both contact dermatitis and atopic dermatitis. Topical application of acetyl carnitine and lipoic acid according to the invention can also be effective to prevent symptoms in aging persons for the inhibition of microscarring of the dermis and to promote collagen production. It is an advantage of the invention that that treatment or preventive measures employ, as an active ingredient, natural compounds. It is another advantage of the invention that topical application of acetyl carnitine and lipoic acid provides a simple, non-invasive, nontoxic, over-the-counter topical method for treating all kinds of skin damage.

### EXAMPLES

The following examples are presented to further illustrate and explain the present invention and should not be taken as limiting in any regard. Unless otherwise indicated, all percentages are by weight. Lipoic acid was supplied by the Henkel Corporation and was placed into a lecithin-based oil-in-water cream at a level of 5%

All references cited herein are hereby incorporated by reference, as are additional ingredients and methods set out in U.S. Pat. Nos. 4,775,530, 5,376,361, 5,409,693, 5,545,398, 5,574,063, 5,643,586, 5,709,868, 5,879,690, 5,965,618, 5,968,618, 6,051,244, 6,191,121, 6,296,861, and 6,365,623. Generally, these compositions contain other active ingredients summarized above that enhance the effect of active ingredients of the invention.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the invention in any sequence which is effective to meet the objectives there intended, unless the context specifically indicates the contrary.

1. A method for the treatment of skin damage comprising topically applying to affected skin areas a composition containing an effective amount of acetyl carnitine and lipoic acid or a lipoic acid derivative.

2. A method according to claim 1 wherein the acetyl carnitine is L-acetyl carnitine.

3. A method according to claim 1 wherein the lipoic acid derivative is selected from the group consisting of dihydrolipoic acid, a lipoic or dihydrolipoic acid ester, a lipoic or dihydrolipoic acid amide, lipoic acid plus, a lipoic or dihydrolipoic acid salt, and mixtures thereof.

4. A method according to claim 1 wherein the composition contains lipoic acid, dihydrolipoic acid, and mixtures thereof.

5. A method according to claim 1 wherein the composition further contains an active ingredient selected from the group consisting of tyrosine, an α-hydroxy acid ingredient, folic acid, a fatty acid ester of ascorbic acid, or mixtures thereof.

6. A method according to claim 5 wherein the α-hydroxy acid is glycolic acid.

7. A method according to claim 5 wherein the fatty acid ester of ascorbic acid is ascorbyl palmitate.

8. A method according to claim 5 wherein the composition comprises from about 0.025% to about 10% by weight adjunct ingredient.

9. A method according to claim 1 wherein the composition comprises from about 0.1% to about 7% by weight lipoic acid or lipoic acid derivative, and from about 0.025% to about 5% by weight acetyl carnitine.

10. A method according to claim 1 wherein the composition comprises from about 0.25% to about 5% lipoic acid or lipoic acid derivative, and from about 0.5% to about 2% acetyl carnitine.

11. A method of treating skin inflammation and aging comprising applying to the skin a composition containing from about 0.1% to about 7% by weight lipoic acid or a lipoic acid derivative, and from about 0.025% to about 5% by weight acetyl carnitine in a dermatologically acceptable carrier.

12. A method according to claim 11 wherein the skin inflammation is observed in a pathological condition selected from the group consisting of dermatitis, xerosis, eczema, rosacea, seborrhea, psoriasis, and thermal and radiation burns.

13. A method according to claim 11 wherein the carrier comprises phosphatidyl-choline.

14. A method according to claim 11 wherein the composition contains from about 0.25% to about 5% lipoic acid or lipoic acid derivative, and from about 0.5% to about 2% by weight acetyl carnitine.

15. A method according to claim 11 wherein the composition further contains from about 0.025% to about 10% by weight of an adjunct ingredient selected from the group consisting of tyrosine, folic acid, ascorbyl palmitate, glycolic acid, and mixtures thereof.

16. A method according to claim 15 wherein the composition contains from about 0.5% to about 7% by weight ascorbyl palmitate.

17. A method according to claim 15 wherein the composition contains from about 0. 1% to about 5% by weight tyrosine.

18. A method according to claim 15 wherein the composition contains from about 1% to about 10% by weight glycolic acid.

19. A method acccording to claim 15 wherein the composition contains from about 0.025% to about 1% by weight folic acid.

20. A method for treating skin damage selected from the group consisting of dermatitis, xerosis, eczema, rosacea, seborrhea, psoriasis, thermal and radiation burns, and aging, comprising applying to affected skin areas a composition containing from about 0.05% to about 2% by weight acetyl carnitine, from about 2% to about 5% lipoic acid, and from about 0.1% to about 5% by weight tyrosine.
